(19) United States
(12) Patent Application Publication
(10) Pub. No.: US 2025/0082267 A1
(43) Pub. Date: Mar. 13, 2025

(54) Flex Force Smart Glove with Photoplethysmography

(71) Applicant: Lasarrus Clinic and Research Center Inc, Middle River, MD (US)

(72) Inventor: Lloyd Emokpae, Baltimore, MD (US)

(21) Appl. No.: 18/798,702

(22) Filed: Aug. 8, 2024

Related U.S. Application Data
(60) Provisional application No. 63/531,436, filed on Aug. 8, 2023.

(51) Int. Cl. A61B 5/00; A61B 5/08; A61B 5/1455

(52) U.S. Cl. CPC A61B 5/6806; A61B 5/0803; A61B 5/0816; A61B 5/14552; A61B 5/726; A61B 2560/0242; A61B 2560/0462

(57) ABSTRACT

A Flex Force Smart Glove with Photoplethysmography provides a wearable health technology for patients with chronic respiratory diseases such as COPD to aid in continuous blood oxygen saturation (SpO2) and respiration rate (RR) monitoring addressing and correcting racial biases of conventional devices by intelligently accounting for variations in skin pigmentation and thickness. Equipped with environmental sensors to detect PM2.5 and PM10 particulate matter, health and environmental monitoring are merged to provide a comprehensive solution to both individual and collective health challenges.

### CROSS-REFERENCES TO RELATED APPLICATIONS

This application claims the benefit of U.S. Provisional Application Ser. No. 63/531,436, filed Aug. 8, 2023, the contents of which are hereby incorporated by reference.

### BACKGROUND OF INVENTION

### Field of the Invention

Chronic Medical Problem: In contemporary healthcare settings, wearable pulse oximeter sensors are widely used tools. These devices provide a non-invasive method to monitor peripheral blood oxygen saturation (SpO2), respiration rate (RR) [20], and environmental particulates PM2.5 and PM10 [21], serving as a surrogate for expensive and invasive direct measurements of arterial blood oxygen saturation (SaO2). SpO2 measurements are critical in diagnosing and managing conditions such as Chronic Obstructive Pulmonary Disease (COPD). However, evidence indicates that current pulse oximeter technology demonstrates racial bias [1-4]. Research shows that pulse oximeters tend to be less accurate for African American, Asian, and Hispanic patients compared to Caucasian patients [1-4][9][10]. This bias has significant implications on patient care, leading to disparities in healthcare outcomes among racial and ethnic minorities. A recent safety warning from the US Food and Drug Administration (FDA) about pulse oximeters' use in minority populations [11] underscores the urgent need to address this critical problem.

The 2023 Canadian wildfires [22]-[26] demonstrated the severity of environmental hazards, with fine particulate matter, PM2.5 and PM10, carried by the smoke posing significant health risks, particularly for sensitive groups including those with respiratory conditions like COPD. Therefore, an integrated system that can monitor exposure to environmental hazards like PM2.5 and PM10, in addition to tracking SpO2 and RR, is critical in managing diseases like COPD.

### Description of Related Art

Current pulse oximeter technology relies on the photoplethysmography technique. However, these sensors cannot accurately differentiate changes in light absorption due to variations in hemoglobin (Hb) to oxyhemoglobin (HbO2) ratio from those due to variations in skin pigmentation [12][13]. This fundamental limitation contributes to disparities in healthcare, notably within the US healthcare system, leading to poorer patient outcomes and higher mortality rates among minority patients [14]. Aspects of the present innovation aim to address this significant gap by developing a novel wearable pulse oximetry system that detects patient skin tone, adjusts for its effects on SpO2 measurements, and tracks PM2.5 and PM10 environmental particulates. This innovative approach can potentially mitigate racial biases inherent in current technologies and promote health equity.

COPD is a common, preventable, and treatable condition, characterized by persistent respiratory symptoms and airflow limitation due to airway and/or alveolar abnormalities [15][16]. This disorder, typically caused by significant exposure to harmful particles or gases, mainly includes two conditions, emphysema and chronic bronchitis, often presenting concurrently [17]. Its management and diagnosis traditionally rely on spirometry, which is considered the gold standard [18]. However, spirometry is not without limitations—it requires substantial patient effort and specialized personnel for the accurate interpretation of results. Adding SpO2, RR, and environmental data to the diagnostic mix could augment the information yielded by spirometry, allowing for a more comprehensive, dynamic, and potentially automated assessment of COPD. In particular, continuous SpO2 monitoring could detect periods of decreased oxygen saturation not noticeable during a standard spirometry test, further enhancing the clinical utility of these devices.

Despite the FDA warning [11], Congressional Inquiry [19], and numerous documented biases [1-4][9][10], a 2022 review of pulse oximeter technology found no attempts by manufacturers to adjust algorithms in pulse oximeters [10]. This technological void extends beyond blood oxygen saturation measurements due to pulse oximeters' widespread use in health monitoring and disease diagnosis. The inventive pulse oximeter technology aligns with the increasing need for diagnostic tools for in-home care and managing the rising rates of cardiorespiratory diseases.

### SUMMARY OF INVENTION

In some aspects, the techniques described herein relate to a wearable photoplethysmography system including: a first photoplethysmogram (PPG) sensor disposed to be worn on a patient's finger; a second PPG sensor disposed to be work on the patient's wrist; a processor configured to continuously estimate RR and SpO2 using data from the first and second sensors; wherein the processor is further configured to adjust for a patient's skin pigmentation and thickness.

In some aspects, the techniques described herein relate to a wearable photoplethysmography system, wherein the system further includes a glove into which the first and second sensors and processor are disposed.

In some aspects, the techniques described herein relate to a wearable photoplethysmography system, wherein the processor is configured to apply inverse synchro squeezed wavelet transform (ISSWT) to the sensor data to derive RR.

### BRIEF DESCRIPTION OF THE DRAWINGS

FIGS. 1A and 1B illustrate embodiments of the present invention on patients' wrist and hands;

FIG. 1C schematically illustrates sensor data flow and processing in accordance with embodiments of the invention;

FIG. 2 illustrates five-day averages of PM2.5 and PM10 levels in Maryland during the Canadian wildfires of summer 2023;

FIG. 3 illustrates SpO2 before pigmentation correction;

FIG. 4 illustrates SpO2 after pigmentation for all patients showing a reduction in error bars;

FIG. 5 illustrates probabilities of exposure during a wildfire event; and

FIG. 6 illustrates a regression model showing varying probabilities of pulmonary exposure during a wildfire (in a bar graph).

### DETAILED DESCRIPTION

Reference will now be made in detail to exemplary embodiments, discussed with regard to the accompanying drawings. In some instances, the same reference numbers will be used throughout the drawings and the following description to refer to the same or like parts. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. For example, unless otherwise indicated, method steps disclosed in the figures can be rearranged, combined, or divided without departing from the envisioned embodiments. Similarly, additional steps may be added or steps may be removed without departing from the envisioned embodiments. Thus, the materials, methods, and examples are illustrative only and are not intended to be necessarily limiting.

### FFSG-PPM Technology

The FFSG-PPM, based on patented wearable flex force smart glove technology (FFSG), see U.S. Pat. No. 10,890,970 by the present inventor (the contents of which are hereby incorporated by reference), represents a transformative step in the evolution of wearable health monitoring. Aspects of the present invention provide a racially inclusive SpO2 monitoring system. Current pulse oximeter technologies, relying heavily on photoplethysmography techniques, often fail to differentiate light absorption changes due to variations in hemoglobin (Hb) to oxyhemoglobin (HbO2) ratio from those arising from skin pigmentation differences. Such limitations have led to significant disparities in healthcare outcomes, particularly within the US healthcare system, resulting in poorer patient outcomes and increased mortality rates among minority patients. Present embodiments of FFSG-PPM's advanced sensing and algorithmic capabilities can address and correct these biases, ensuring equitable and accurate SpO2 monitoring across diverse populations.

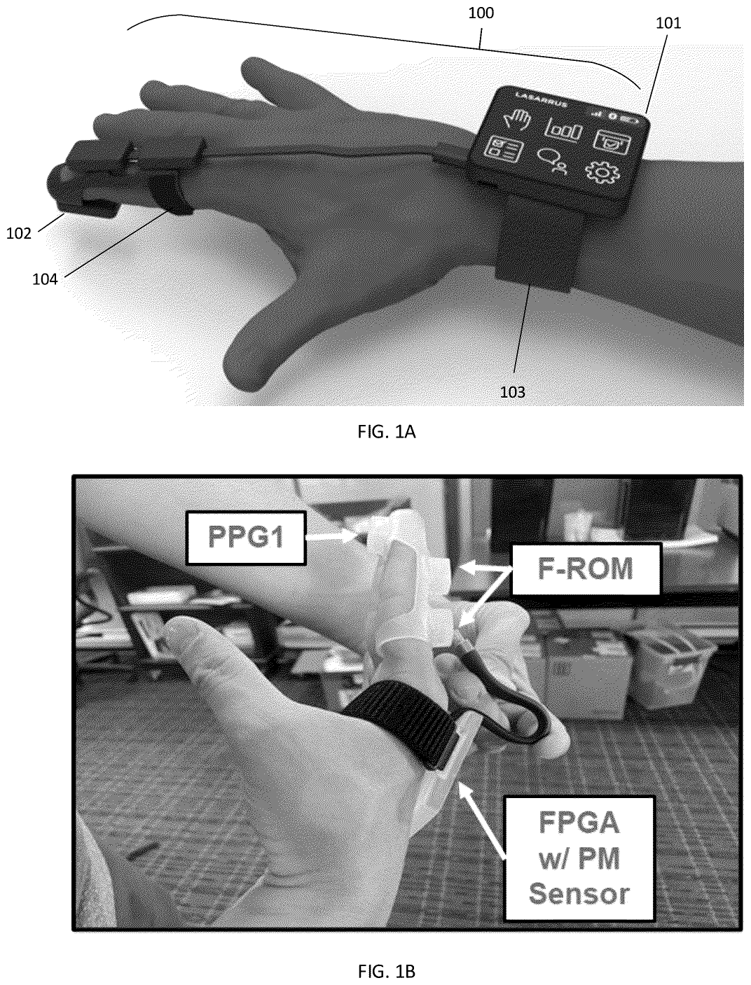

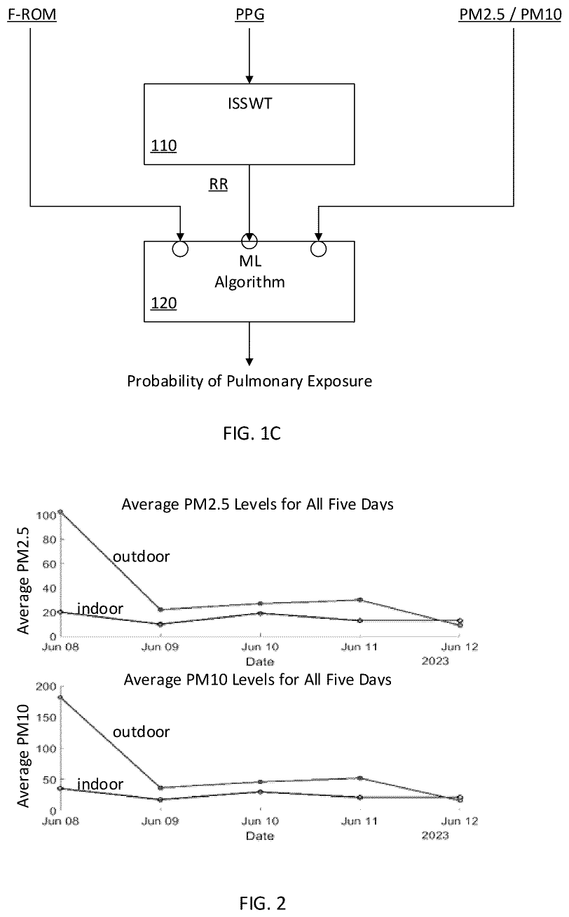

In one embodiment, a glove integrates two PPG sensors-one placed on an index finger for example and the other embedded within the glove's main processor. This dual-sensor configuration facilitates comprehensive data capture, minimizing chances of inaccuracies. In embodiments, an inverse synchro squeezed wavelet transform (ISSWT) technique processes SpO2 time series data, refining it to accurately derive an RR. Such advanced computational techniques ensure that the data's integrity is maintained, and readings are precise. In some embodiments, sensors 101 and 102 are mounted on wrist band 103 and finger band 104, respectively, as illustrated in FIG. 1A.

Embodiments of the FFSG-PPM such as exemplary embodiment 100 may also incorporate environmental sensors. With events like the 2023 Canadian wildfires highlighting significant health risks posed by particulates such as PM2.5 and PM10 particulates, especially to those with pre-existing respiratory conditions like COPD, the need for real-time environmental monitoring has never been more pressing. FFSG-PPM sensors can capture these particulate levels and offer users a holistic view of both their health and the environment around them.

In embodiments, the FFSG-PPM may be not just a wearable health monitor, but a comprehensive health companion, tailored to meet the unique challenges of the times. By bridging the gaps in current technologies and integrating environmental monitoring, it stands as a beacon of innovation in the realm of wearable health tech.

### Innovation and Invention

In embodiments, the FFSG-PPM may provide a vision for a more inclusive and comprehensive healthcare future. Inventive design elements, from the dual PPG sensor configuration to sophisticated algorithms enable precision. In embodiments including integrated sensors that monitor real-time environmental factors, broader health challenges posed by urbanization and industrialization can be addressed.

Six aspects of embodiments of the invention include:
Racially Inclusive SpO2 Monitoring System: Wearable FFSG systems may include a PPG sensor for precise SpO2 and RR estimation in which the system is configured to account for skin pigmentation and thickness, eliminating the racial bias in SpO2 monitoring and ensuring accurate readings for all skin tones. As illustrated in FIGS. 1A, 1B, and 1C, a smart glove in accordance with embodiments of the invention includes two PPG sensors: one on the index finger and another integrated into the main glove processor. The dual PPG sensor design allows for increased accuracy and redundancy, as pulse oximetry readings taken from the finger are generally more accurate than those taken from the wrist due to stronger and more consistent blood flow [29][30][31]. The main glove processor may apply advanced processing 110, such as the inverse synchro squeezed wavelet transform (ISSWT), to convert the SpO2 time series to the frequency domain and then determine the number of breaths per minute, or the respiration rate (RR).
Environmental Exposure Monitoring: In embodiments, the system may also integrate environmental air quality sensors to measure particulate matter (PM2.5 and PM10 for example), enabling real-time monitoring of environmental hazards and providing critical data for managing respiratory illnesses like COPD. FIG. 2 illustrates a five-day average of PM2.5 and PM10 levels during a wildfire event, demonstrating how environmental data can be used in conjunction with SpO2 and finger motion to determine the probability of pulmonary exposure.
Machine Learning Algorithms for Bias Compensation: In embodiments, an advanced machine learning algorithm 120 may compensate for the impact of skin pigmentation on PPG signals and variations in skin thickness, thereby ensuring accurate, continuous SpO2 monitoring for all patients, regardless of skin color.
Wearable Form Factor: In some embodiments, the system is in the form of a smart glove, which is easy to wear and provides continuous real-time physiological status monitoring (RT-PSM), crucial for patients with chronic respiratory illnesses. The design can potentially be adapted into different forms suitable for various use cases and patient comfort.
Integration with Digital Health and AI Tools: Coupled with digital health and artificial intelligence tools, embodiments of the invention facilitate continuous, real-time monitoring, improving the assessment and intervention for chronic respiratory conditions. For risk assessment, embodiments implement a regression mechanism based on recurrent neural network (RNN) in order to predict the probability of pulmonary exposure.
Standalone Device for Ease of Use: Embodiments of the invention may provide a standalone device not requiring additional training by clinicians, allowing seamless integration with current clinical and healthcare procedures. In some embodiments, the main sensor unit is based on an Intel-Arm FPGA design that allows for in-situ implementation of advanced signal processing and machine learning algorithms for low-power and real-time computation without requiring a smartphone.

Embodiments of the PPG sensor architecture integrate three sensing modules: a dual-wavelength light emitter and detector for SpO2 and RR measurements, a colorimetry sensor for detecting skin tone, and environmental sensors for detecting particulates such as PM2.5 and PM10. Machine learning algorithms may model and compensate for the effects of skin pigment and thickness on SpO2 measurements, thus improving accuracy and equity compared to the current standard of care.

### Preliminary Evidence

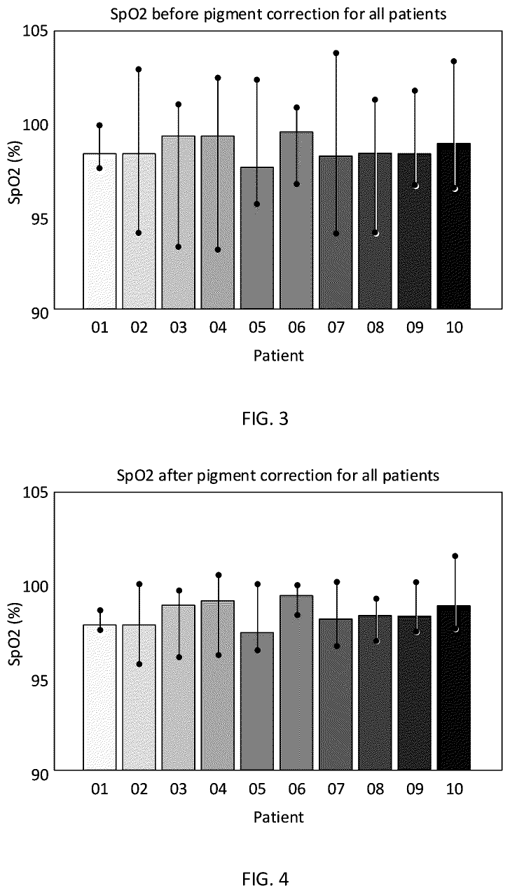

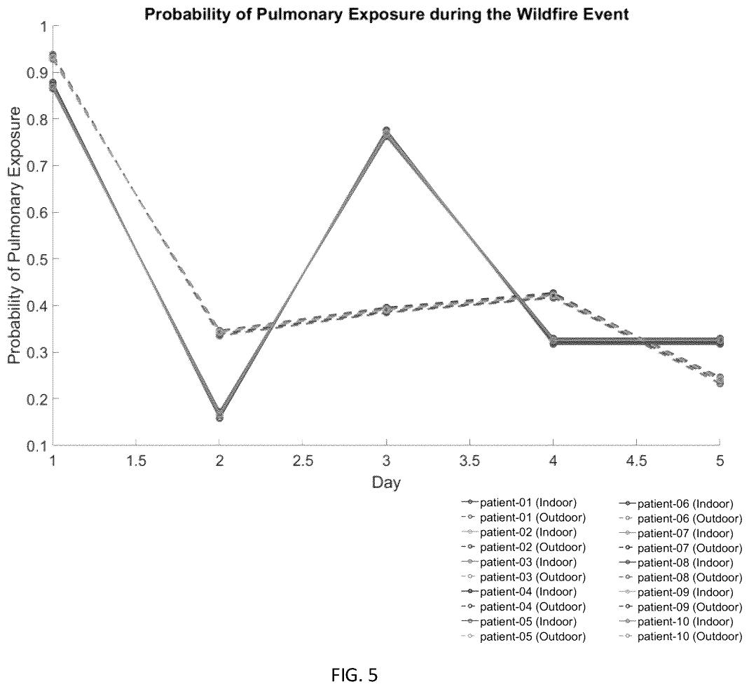

In a prototype system, aspects were designed based on similar sensor technology used in the Physionet BIDMC PPG and Respiration Dataset [32]. Initial feasibility was demonstrated to include preliminary validation of the processing steps for skin pigmentation correction and calculating the probability of pulmonary exposure during wildfire events. Initial data collected from ten patients, utilizing the principles of the BIDMC dataset, provided a foundation for a skin pigmentation correction process. Raw SpO2 measurements before pigment correction exhibited variability across patients, with average SpO2 values ranging from 97.62% to 99.55%. The confidence intervals for these measurements demonstrated substantial variability, reflecting the inherent variability in the raw SpO2 measurements. Applying the pigment correction to these measurements led to subtle yet crucial changes. For instance, patient-02's average SpO2 shifted slightly from 98.29% pre-correction to 97.84% post-correction. Significantly, the 95% confidence interval for patient-02 tightened substantially, indicating a reduction in variability and demonstrating the potential effectiveness of the correction in mitigating the impact of skin pigmentation on SpO2 measurements. These results, summarized in FIGS. 3 and 4, strongly suggest the potential benefits of applying a pigment correction to SpO2 measurements, increasing the reliability of these measurements across diverse populations.

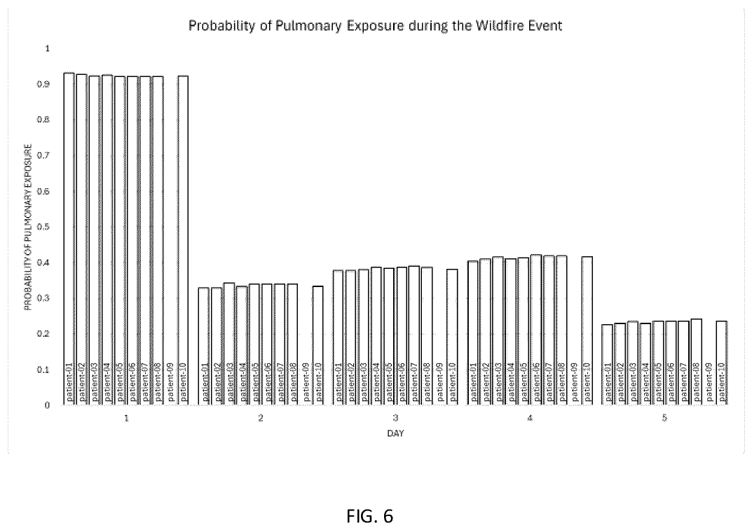

In addition to skin pigmentation correction, computations of the probability of pulmonary exposure during wildfire events, as summarized in FIG. 5, have been demonstrated. This process involved collecting patient-specific physiological data (HR and RR) and environmental data (PM2.5 and PM10 levels). A logistic regression model was then employed to calculate the probability of pulmonary exposure for each day during the wildfire event. Initial analysis of the data reveals that the probabilities of pulmonary exposure varied considerably across patients and were consistently higher for outdoor exposure than indoor exposure, suggesting that outdoor conditions during wildfire events posed a higher risk for pulmonary exposure. FIG. 6 illustrates varying probabilities of pulmonary exposure during a wildfire based on a regression model.

Taken together, these results highlight the utility of the inventive advanced wearable photoplethysmography sensor system.

### Additional Information—Linear Models

The equation for the linear correction, which is derived from Bickler's pigmentation correction [33], is described as:

$$SpO {2}_{corrected} = SpO {2}_{Raw} - k \cdot ( {pigment}_{index} - {pigment}_{reference} ) - Ω \cdot ( 100 - SaO 2 ) \qquad ( 1 )$$

where k the pigment correction factor determined empirically, Ω is the arterial saturation correction factor (during model training), the pigment index is derived from the colorimetric sensor measurements, and the reference pigment index is a standard value derived from a reference population. To improve SpO2 accuracy, acoustics may be incorporated and a linear regression-based ML model may be used to adjust for skin pigmentation effects. Exponential, logarithmic, or non-linear models may also be utilized. Ultrasound (acoustic) sensing can be used to compensate for variations in optical readings caused by different skin colors and measure arterial stiffness via pulse wave velocity (PWV) [34][35], by adjusting the pigment index. This approach can enhance SpO2 measurement accuracy by assessing arterial stiffness and blood flow dynamics.

In addition, a key innovation of the EquiPulse system is the ability to provide quantifiable environmental exposure risks assessment for patients with COPD through a parameter, called the probability of pulmonary exposure (PPE). A PPE metric is initially determined using a linear regression model derived from the data obtained in a preliminary analysis that integrates heart rate (HR), respiration rate (RR), and particulate matter levels (PM2.5 or PM10) as follows:

$$P P E = {w}_{1} \cdot \frac{H R - min ( H R )}{max ( H R ) - min ( H R )} + {w}_{2} \cdot \frac{R R - min ( R R )}{max ( R R ) - min ( R R )} + {w}_{3} \cdot \frac{P M - min ( P M )}{max ( P M ) - min ( P M )} \qquad ( 2 )$$

where w₁, w₂, and w₃ are weights that can be determined empirically from recorded data during model training. Non-linear models may also be representative of the relationships. Changes in air quality may alter PPE, which will correlate with spirometry measures like FEV1 in COPD patients

### Additional Information—Sensor Design

1. Finger Circuit Design with Ultrasonic Sensors: Some embodiments may include a compact finger sensor board of approximately 1.52 cm², integrating the MAX30101EFD+ chip for reliable PPG & HR sensing, Vishay VEML3328 colorimetry sensor, and the ICM-20948 unit for precise 3-axis finger motion detection. This motion sensor aids in compensating for motion artifacts, enhancing the signal-to-noise ratio, and supporting advanced algorithms that filter out noise due to finger movements, thereby improving SpO2 accuracy and the reliability of readings even during finger motions. The board can employ SMT ZIF connectors for efficient interfacing with the main processor board. This includes ultrasonic transducer elements on the same printed circuit board as the PPG and colorimetry sensors. In some embodiments, a 10 MHz ultrasonic transducer system, comprising a transmitter and receiver, with a form factor of approximately 30 mm in length, 15 mm in width, and 5 mm in height, can measure pulse wave velocity (PWV) and determine arterial stiffness. This transducer system can be integrated into embodiments and utilized in the ML model to enhance SpO2 measurement accuracy by assessing arterial stiffness and blood flow dynamics.

2. Main Processor Board Design: In some embodiments, a more extensive PCB (e.g., ˜4.72 cm²) is used for the hand/wrist region. In some embodiments, this board may be powered by the Intel-Terasic SoC System on Module, with dual-core ARM Cortex-A9 CPU and 110K FPGA Logic Elements. ZIO-101962/ZIO-101963 sensors may be incorporated for real-time particulate (e.g., PM2.5 and PM10) monitoring, and a Terasic LT24 LCD may be included for intuitive data display. A Digi Xbee 3 module may be integrated into a wireless mesh and Bluetooth technology, providing robust wireless communication capabilities, facilitating seamless over-the-air (OTA) updates, integration with future centralized monitoring stations, and enabling real-time data transmission to clinicians. In embodiments, both the finger sensor and main processor boards can be integrated into a wearable glove. Initial lab verification tests can focus on the system's ability to reliably measure and display raw sensor values, such as SpO2Raw, raw colometry values, raw PWV from the ultrasonic system and using [34], and raw PM2.5 and PM10 values (calibrated using, for example, a Temtop PMD 351 commercial PM system).

### Additional Information—ML Design

1. ML Algorithm SpO2 Correction: A Recurrent Neural Network (RNN) based ML model may be used to optimize the coefficients for the SpO2 compensation used in Eq (1). The input to the model may include (a) the derived PWV computed from the co-located ultrasonic sensors around the finger and wrist, (b) the colorimetric sensor measurement, (c) reference pigment, and (d) reference SaO2 value (only during training). The output from the model may be the optimized correction coefficients used in Eq (1) to accurately determine SpO2. The RNN incorporates sensor positions and localization accuracy to filter the input layer of the spatio-temporal Long Short-Term Memory (LSTM).

2. ML Algorithm PPE: Running in parallel to the SpO2 correction above, another RNN ML model may be used to determine the best coefficients for the probability pulmonary exposure used in Eq (2). The input to the model may include (a) the HR determined from the SpO2 measurement, (b) the RR also determined from SpO2, and (c) the particulate matter levels (PM2.5 or PM10). The output may be the optimized coefficients used in Eq (2) to accurately determine PPE.

3. ML Model Training: Using the training set (N_{TRAIN}=60) from the 100 COPD subjects with diverse skin tones recruited for the study, up to 120 hours of data may be obtained (2 hrs. per COPD patient) during an in-clinic visit. 70% of the collected data may be allocated for ML training (84 hours of recording) and 30% of the collected data for ML testing (36 hours), which consistent with common approach in the field. ML models may be trained with the data collected from the EquiPulse prototype system. A model's performance may be assessed based on its accuracy, sensitivity, and specificity on the test set.

It is appreciated that certain aspects of the above-described embodiments can be implemented by hardware, or software, or a combination of hardware and software. If implemented by software, it can be stored in tangible computer-readable media. The software, when executed by the processor can perform disclosed method steps.

Consistent with the present disclosure, a processor in a patient device 220, a sensor node processor 106, and a processor within a server 230 may be configured with machine learning algorithms in order to implement any of the systems and methods disclosed herein. In some embodiments, machine learning algorithms (also referred to as machine learning models) may be trained using training data. Some non-limiting examples of such machine learning algorithms may include classification algorithms, and data regressions algorithms. In embodiments, a trained machine learning algorithm may include an inference model, such as a predictive model, a classification model, and a regression model.

### REFERENCES

[1] Crooks, C. J.; West, J.; Morling, J. R.; Simmonds, M.; Juurlink, I.; Briggs, S.; Cruickshank, S.; Hammond-Pears, S.; Shaw, D.; Card, T. R.; et al. Pulse oximeters' measurements vary across ethnic groups: An observational study in patients with Covid-19 infection. Eur. Respir. J. 2022, 2103246, doi:10.1183/13993003.03246-2021.
[2] Valbuena, Valeria S. M., Raina M. Merchant, and Catherine L. Hough. “Racial and Ethnic Bias in Pulse Oximetry and Clinical Outcomes.” JAMA Internal Medicine 182.7 (2022): 699-700
[3] Valbuena, Valeria S. M., Sarah Seelye, Michael W. Sjoding, Thomas S. Valley, Robert P. Dickson, Steven E. Gay, Dru Claar, Hallie C. Prescott, and Theodore J. Iwashyna. “Racial bias and reproducibility in pulse oximetry among medical and surgical inpatients in general care in the Veterans Health Administration 2013-19: multicenter, retrospective cohort study.” BMJ 378 (2022): e069775.
[4] Sjoding, Michael W., Robert P. Dickson, Theodore J. Iwashyna, Steven E. Gay, Dru Claar, Hallie C. Prescott, and Thomas S. Valley. “Racial Bias in Pulse Oximetry Measurement.” The New England Journal of Medicine 384 (2021): 246-248
[5] “Pulse Oximeter Market Size, Share, Growth|Global Report [2029].” Fortune Business Insights, 2023, www.fortunebusinessinsights.com/pulse-oximeter-market-102326.
[6] “Pulse Oximeter Market Size, Share & Trends Analysis Report By Product (Fingertip, Handheld), By End-use (Hospitals & Clinics, Homecare), By Region, And Segment Forecasts, 2021-2028.” Grand View Research, 2023, www.grandviewresearch.com/industry-analysis/pulse-oximeters-market.
[7] “Pulse Oximeter Market by Product (Equipment, Sensor), Type (Portable, Handheld), End User (Hospital Care, Home Care), COVID-19 Impact—Global Forecast to 2026.” MarketsandMarkets™ INC., 2023, www.marketsandmarkets.com/Market-Reports/pulse-oximeters-market-241669663.html.
[8] “Remote Patient Monitoring Market Size Worth $62 Billion By 2028|CAGR: 12.5%.” Grand View Research, 2023, www.grandviewresearch.com/press-release/global-remote-patient-monitoring-market.
[9] Wong, A. K. I.; Charpignon, M.; Kim, H.; Josef, C.; De Hond, A. A. H.; Fojas, J. J.; Tabaie, A.; Liu, X.; Mireles-Cabodevila, E.; Carvalho, L.; et al. Analysis of Discrepancies Between Pulse Oximetry and Arterial Oxygen Saturation Measurements by Race and Ethnicity and Association With Organ Dysfunction and Mortality. JAMA Netw. Open 2021, 4, e2131674-e2131674, doi:10.1001/JAMANETWORKOPEN.2021.31674.
[10] Tobin, M. J.; Jubran, A. Pulse oximetry, racial bias and statistical bias. Ann. Intensive Care 2022, 12, 1-2, doi:10.1186/S13613-021-00974-7/METRICS.
[11] Pulse Oximeter Accuracy and Limitations: FDA Safety Communication|FDA Available online: www.fda.gov/medical-devices/safety-communications/pulse-oximeter-accuracy-and-limitations-fda-safety-communication.
[12] Mannheimer, P. D.; Casciani, J. R.; Fein, M. E.; Nierlich, S. L. Wavelength selection for low-saturation pulse oximetry. IEEE Trans. Biomed. Eng. 1997, 44, 148-158, doi:10.1109/10.554761.
[13] Shi, Chunhu et al. “The accuracy of pulse oximetry in measuring oxygen saturation by levels of skin pigmentation: a systematic review and meta-analysis.” BMC medicine vol. 20.1 267. 16 Aug. 2022, doi:10.1186/s12916-022-02452-8
[14] Fawzy, Ashraf, et al. “Racial Disparities in Pulse Oximeter Device Performance.” American Journal of Epidemiology, vol. 192, no. 5, 2023, pp. 703-711.
[15] “CHAPTER 1: DEFINITION AND OVERVIEW.” Global Initiative for Chronic Obstructive Lung Disease, 2021, goldcopd.org/wp-content/uploads/2021/05/GOLD-2021-Chapter-1.pdf.
[16] GBD Chronic Respiratory Disease Collaborators. “Prevalence and attributable health burden of chronic respiratory diseases, 1990-2017: a systematic analysis for the Global Burden of Disease Study 2017.” The Lancet. Respiratory medicine vol. 8.6 (2020): 585-596. doi:10.1016/S2213-2600(20)30105-3.
[17]“COPD—Symptoms and Causes.” Mayo Clinic, mayoclinic.org/diseases-conditions/copd/symptoms-causes/syc-20353679.
[18]“GOLD Spirometry Guide.” Global Initiative for Chronic Obstructive Lung Disease, goldcopd.org/gold-spirometry-guide/.
[19]“Pulse Oximeter Performance, Racial Inequity, and the Work Ahead.” Respiratory Care, vol. 67, no. 2, 2022, pp. 252-254.
[20] Apple Watch Series 7, www.apple.com/watch/compare/.
[21] Atmotube. “Atmotube PRO: Portable Air Quality Monitor.” Atmotube, 2023.
[22]“Nearly 100 million in Northeast, Mid-Atlantic still trapped under Canadian wildfire smoke.” Fox Weather. 7 Jul. 2023. Web.
[23]“Latest Canadian wildfire smoke maps show where air quality is unhealthy now and forecasts for the near future.” CBS News. 7 Jul. 2023. Web.
[24] Cappucci, Matthew and Samenow, Jason. “Canadian wildfires cause air quality alerts as smoke hits U. S.” The Washington Post. 7 Jun. 2023. Web.
[25]O'Kane, Caitlin. “New York City's air pollution among the world's worst as Canada wildfires rage.” CNN. 6 Jun. 2023. Web.
[26]“Maps, satellite images show Canadian wildfire smoke affecting air quality across U. S.” CBS News. 11 May 2023. Web.
[27] Flex Force Smart Glove for Measuring Sensorimotor Stimulation, U.S. Pat. No. 10,890,970, issued Jan. 12, 2021 (Lloyd Emokpae, inventor).
[28] Intel Developer Zone, Flex Force Smart Glove, https://software.intel.com/en-us/articles/flex-force-smart-glove-use-intel-soc-fpga-to-measure-sensorimotor-data
[29] Medical News Today. “Pulse oximetry: Uses and benefits.” Medical News Today, 2023.
[30] Personal Health Monitors. “Oximeter Readings Different On Each Hand.” Personal Health Monitors, 2023.
[31] Geeky Medics. “Pulse Oximetry: Uses and Limitations.” Geeky Medics, 2023.
[32] Goldberger, A., Amaral, L., Glass, L., Hausdorff, J., Ivanov, P. C., Mark, R., . . . & Stanley, H. E. (2000). PhysioBank, PhysioToolkit, and PhysioNet: Components of a new research resource for complex physiologic signals. Circulation [Online]. 101 (23), pp. e215-e220.)
[33] Philip E. Bickler, John R. Feiner, John W. Severinghaus; Effects of Skin Pigmentation on Pulse Oximeter Accuracy at Low Saturation. Anesthesiology 2005; 102:715-719 doi: https://doi.org/10.1097/00000542-200504000-00004
[34] Jonathan Vappou, Jianwen Luo, Elisa E. Konofagou, Pulse Wave Imaging for Noninvasive and Quantitative Measurement of Arterial Stiffness In Vivo, American Journal of Hypertension, Volume 23, Issue 4, April 2010, Pages 393-398, https://doi.org/10.1038/ajh.2009.272
[35] Chengwu Huang, Yuan Su, Hong Zhang, Lin-Xue Qian and Jianwen Luo, “Pulse wave velocity measurement in healthy and diseased carotid arteries in vivo,” 2015 IEEE International Ultrasonics Symposium (IUS), Taipei, 2015, pp. 1-4, doi: 10.1109/ULTSYM.2015.0003.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of at least one other feature, integer, step, operation, element, component, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A wearable photoplethysmography system comprising:
a first PPG sensor disposed to be worn on a patient's finger;
a second PPG sensor disposed to be work on the patient's wrist; and
a processor configured to continuously estimate RR and SpO2 using data from the first and second sensors;
wherein the processor is further configured to adjust for a patient's skin pigmentation and thickness.

2. The wearable photoplethysmography system of claim 1 further comprising a glove into which the first and second sensors and processor are disposed.

3. The wearable photoplethysmography system of claim 1, wherein the processor is configured to apply inverse synchro squeezed wavelet transform (ISSWT) to the sensor data to derive RR.

4. The wearable photoplethysmography system of claim 1, further comprising an integrated environmental sensor for detection of environmental atmospheric particulates.

5. The wearable photoplethysmography system as claimed in claim 2, wherein the processor is configured to apply inverse synchro squeezed wavelet transform (ISSWT) to the sensor data to derive RR.

6. The wearable photoplethysmography system as claimed in claim 2, further comprising an integrated environmental sensor for detection of environmental atmospheric particulates.

7. The wearable photoplethysmography system as claimed in claim 3, further comprising an integrated environmental sensor for detection of environmental atmospheric particulates.

8. The wearable photoplethysmography system as claimed in claim 5, further comprising an integrated environmental sensor for detection of environmental atmospheric particulates.
